(19) United States
(12) Patent Application Publication
(10) Pub. No.: US 2005/0260284 A1
(43) Pub. Date: Nov. 24, 2005

(54) Juvenile weight reduction formula

(76) Inventor: Giovina DiMateeo-Leggio, Aventura, FL (US)

Correspondence Address: GIOVINA INTERNATIONAL INC. OF AMERICA, SUITE #807, 19495 BISCAYNE BLVD., AVENTURA, FL, 33180, US

(21) Appl. No.: 10/628,344

(22) Filed: May 21, 2004

(51) Int. Cl. A61K 35/78

(52) U.S. Cl. 424/725

(57) ABSTRACT

The supplements are a combination of is a healthy and effective fiber based formula to that helps control carbohydrate cravings, reduces hunger significantly, lowers serum cholesterol and glucose, burns fat, builds muscle mass and results with healthy weight loss. They are packaged in bottles of 180 chewable tablets. The supplements are in the form of wafers of which three tablets are taken orally twice daily one hour before lunch and one hour before dinner.

Healthy weight loss is achieved when the fiber based supplement is taken before meals with a full glass of water, expanding in the stomach giving a feeling of fullness. As a result, less hunger and more controlled eating. The chromium helps reduce cravings for carbohydrates while reducing cholesterol, burning fat and building muscle.

Glucomannan (Konjac Root) is a high fiber supplement taken from the Konnyaku root. Glucomannan is an effective weight control agent that helps to reduce cholesterol, normalizes blood sugar and absorbs toxic substances produced during digestion and elimination of waste. Glucomannan has the ability to absorb water and swell to 50 times its original volume creating a full feeling. Some studies have reported improved glucose control and decreased cholesterol. Glucomannan is a natural dietary, calorie free, high fiber powder which is obtained from the root of the Amorphophallus Konjac, which has been safely consumed as food for over 1000 years in the Orient. It is a pectin-like, gel fiber which absorbs liquid (up to 50 times its weight) giving a feeling of fullness. Studies of human subjects and rats have indicated that glucomannan forms a gel and greatly increases the moisture content of the food bolus during digestion. Glucomannan promotes bowel health and helps to relieve stress on the pancreas. A study published in the Journal of Obesity (1984) 8,289293 conducted by David E. Walsh, Vazgen Yaghoubian and Ali Behforooz, stated that Glucomannan forms a gel around the food particles, causing digestive enzymes to release sugars and fats at a slow rate. This process greatly increases the moisture content of the food bolus during digestion.

Studies indicate that our Weight Reduction Formula containing chromium picolinate helps reduce carbohydrate cravings, lower serum cholesterol and glucose, burn fat, and build muscle. Chromium regulates insulin function. If you aren't getting enough chromium, your insulin cannot perform as effectively as possible. Rather than moving glucose into your cells to be burned for energy, it remains in your blood stream and is more likely to be stored as fat.

A double blind, crossover study showed an average of 7% cholesterol reduction in subjects taking chromium picolinate, enhances the ability of insulin to lower serum glucose levels; can also produce anabolic effects similar to steroids, but without any of the side effects. It not only increases strength and musde mass, but was also shown to decrease body fat by 22% in one study.

1. I claim that our Juvenile Weight Reduction Formula is a healthy and effective fiber based formula to that helps implement a healthy lifestyle, controls carbohydrate cravings, reduces hunger significantly, lowers serum cholesterol and glucose, burns fat, builds muscle mass and results with healthy weight loss.
